(19) United States
(12) Patent Application Publication
(10) Pub. No.: US 2024/0415650 A1
(43) Pub. Date: Dec. 19, 2024

(54) ADJUSTABLE ANNULOPLASTY DEVICE WITH ALTERNATING PEAKS AND TROUGHS

(71) Applicant: Edwards Lifesciences Innovation (Israel) Ltd., Caesarea (IL)

(72) Inventors: Meir Kutzik, Kfar Saba (IL); Haim Brauon, Beit Dagan (IL); Michael Levin, Carmi Yosef (IL); Alon Fogel, Tel Aviv-Yafo (IL); Ilia Hariton, Zichron Yaacov (IL); Tal Reich, Moledet (IL)

(21) Appl. No.: 18/818,519

(22) Filed: Aug. 28, 2024

Related U.S. Application Data
(63) Continuation of application No. 17/213,112, filed on Mar. 25, 2021, now Pat. No. 12,102,533.
Application No. 17/213,112 is a continuation of application No. 16/261,975, filed on Jan. 30, 2019, now Pat. No. 10,959,845.
Application No. 16/261,975 is a continuation of application No. 15/475,871, filed on Mar. 31, 2017, now Pat. No. 10,226,342.

(30) Foreign Application Priority Data
Jul. 8, 2016 (GB) GB1611910.9

(51) Int. Cl. A61F 2/24

(52) U.S. Cl. CPC A61F 2/2445; A61F 2220/0016; A61F 2220/0091; A61F 2250/0006; A61F 2250/0008; A61F 2250/0012; A61F 2250/0029

(57) ABSTRACT

An annuloplasty implant is assembled at a valve of a heart of a subject by (1) transfemorally delivering, to the heart, a first subunit of the implant; and securing the first subunit to tissue of the heart by driving, into the tissue, a first anchor connected to the subunit by a flexible strip; and (2) subsequently, for multiple successive subunits of the implant, iteratively: (a) transfemorally delivering, to the heart, the successive subunit; and (b) securing the successive subunit to the tissue of the heart by driving, into the tissue, a respective anchor connected to the successive subunit by a respective flexible strip. The annuloplasty implant is subsequently contracted to contract the valve. Other embodiments are also described.

### CROSS-REFERENCE TO RELATED APPLICATIONS

The present application is a Continuation of U.S. Ser. No. 17/213,112 to Kutzik et al., filed Mar. 25, 2021, and entitled “Adjustable annuloplasty device with alternating peaks and troughs,” which published as US 2021/0212827;
which is a Continuation of U.S. Ser. No. 16/261,975 to Kutzik et al., filed Jan. 30, 2019, and entitled “Adjustable annuloplasty device with alternating peaks and troughs,” which published as US 2019/0159898 (now U.S. Pat. No. 10,959,845);
which is a Continuation of U.S. Ser. No. 15/475,871 to Kutzik et al., filed Mar. 31, 2017, and entitled “Adjustable annuloplasty device with alternating peaks and troughs,” which published as US 2018/0008409 (now U.S. Pat. No. 10,226,342);
which claims priority from UK Patent Application GB1611910.9, filed Jul. 8, 2016, and entitled “Adjustable annuloplasty device with alternating peaks and troughs,” which is incorporated herein by reference.

### FIELD OF THE INVENTION

The present invention relates in general to valve repair. More specifically, the present invention relates to repair of a cardiac valve of a patient using an adjustable implant.

### BACKGROUND

Dilation of the annulus of atrioventricular heart valves, such as the mitral valve, prevents the valve leaflets from coapting effectively when the valve is closed, thereby resulting in regurgitation of blood from the ventricle through the valve into the atrium. Annuloplasty is a known surgical technique for treatment of a dilated valve annulus. U.S. Pat. No. 9,180,005 to Lashinski et al., which is incorporated herein by reference, relates to an adjustable mitral valve ring for minimally-invasive delivery.

### SUMMARY OF THE INVENTION

Annuloplasty implants are described, which are configured to be percutaneously (e.g., transluminally) delivered to the heart, and adjusted in order to reshape the valve annulus. The anchors comprise a ring and tissue anchors for anchoring the ring to the valve annulus. For some applications, the implants facilitate deflection or pivoting of the tissue anchors with respect to the ring. For some applications, the implants comprise a plurality of subunits that are individually advanceable and anchorable to the valve annulus. Once at the valve, the subunits are connected to form a ring, and the ring is adjusted to reshape the valve annulus.

There is therefore provided, in accordance with an application of the present invention, apparatus for use at a valve of a heart of a subject, the apparatus including:
a ring, including a plurality of struts arranged in a pattern of alternating peaks and troughs,
   each strut having a first end-portion and a second end-portion,
   each peak defined by convergence of adjacent first end-portions disposed at an angle with respect to each other, and
   each trough defined by convergence of adjacent second end-portions; and
a plurality of anchors, each anchor:
   having a longitudinal axis,
   configured to be driven along the longitudinal axis into tissue of the heart,
   coupled to the ring at a respective trough in a manner that facilitates:
      movement of the anchor along the longitudinal axis with respect to the trough, and
      deflection of the longitudinal axis with respect to the trough.

In an application, the apparatus further includes at least one anchor driver, couplable to the plurality of anchors, and configured to anchor the ring to the heart by moving each anchor along its longitudinal axis with respect to its respective trough.

In an application:
the ring further includes a plurality of adjustment elements, and
each adjustment element is movably coupled to respective adjacent first end-portions such that movement of the adjustment element with respect to the adjacent first end-portions changes the angle at which the adjacent first end-portions are disposed with respect to each other.

In an application, the apparatus further includes at least one adjustment tool, reversibly couplable to the plurality of adjustment elements, and configured to move each adjustment element with respect to its respective adjacent first end-portions.

In an application, the adjustment tool is configured to rotate each adjustment element with respect to its respective adjacent first end-portions.

In an application, each adjustment element circumscribes both of the respective adjacent first end-portions.

In an application, the apparatus further includes at least one adjustment tool, reversibly couplable to the plurality of adjustment elements, and configured to rotate each adjustment element around both of its respective adjacent first end-portions.

In an application:
the ring includes a plurality of hinges,
at least one hinge of the plurality of hinges is disposed at each trough, and
at each trough, the respective anchor is coupled to the ring via the at least one hinge.

In an application, each hinge couples the adjacent second end-portions to each other.

In an application, each hinge is a flexure bearing.

In an application, each hinge includes a fabric.

In an application, each anchor is shaped and rotatably coupled to the ring at the respective trough such that rotation of the respective anchor with respect to the ring moves the anchor along its longitudinal axis with respect to the trough.

In an application:
the ring includes a plurality of anchor mounts,
at each trough:
   a respective anchor mount of the plurality of anchor mounts is articulatably coupled (i) to one second end-portion via a first hinge of the plurality of hinges, and (ii) to another second end-portion via a second hinge of the plurality of hinges, and
   the respective anchor is rotatably coupled to the respective anchor mount.

In an application, at each trough the respective anchor is rotatably coupled to the respective anchor mount such that rotation of the respective anchor with respect to the respective anchor mount moves the anchor along its longitudinal axis with respect to the anchor mount.

There is further provided, in accordance with an application of the present invention, a method for use with a valve of a heart of a subject, the method comprising:
transfemorally delivering a plurality of subunits to the heart, each of the subunits including a pair of struts that includes a first strut and a second strut, each strut of the pair having a first end-portion and a second end-portion, each of the subunits defining a trough at which the second end-portion of each strut of the pair is coupled to the second end-portion of the other strut of the pair;
for each subunit, anchoring the trough to tissue that surrounds the valve by driving a tissue anchor into the tissue;
securing the first end-portion of the second strut of a first subunit to the first end-portion of the first strut of a second subunit such that the secured first end-portions converge at an angle to define a peak; and
subsequently to the steps of anchoring and securing, reducing the angle of each peak by actuating a respective adjustment element.

In an application, transfemorally delivering the plurality of subunits to the heart includes:
transfemorally delivering the first subunit to the heart while a longitudinal guide member is coupled to the first-end portion of the second strut of the first subunit;
transfemorally delivering the second subunit to the heart; and
subsequently, guiding the second subunit toward the first subunit by sliding the first end-portion of the first strut of the second subunit over and along the longitudinal guide member to the first-end portion of the second strut of the first subunit.

In an application, the method includes forming a ring from the plurality of subunits.

In an application, actuating the respective adjustment element includes rotating the respective adjustment element with respect to the respective peak.

In an application, the method further includes, subsequently to the step of anchoring, coupling the respective adjustment elements to the first end-portions that define each peak.

In an application, coupling the respective adjustment elements to the first end portions that define each peak includes coupling the respective adjustment elements to the first end portions that define each peak subsequently to securing the step of securing.

The present invention will be more fully understood from the following detailed description of applications thereof, taken together with the drawings, in which:

### BRIEF DESCRIPTION OF THE DRAWINGS

FIGS. 1A-D are schematic illustrations of a prior art implant for use at a valve of a heart of a subject, in accordance with some applications of the invention;

FIGS. 2A-B, 3A-B, and 4 are schematic illustrations of respective implants for use at a valve of a heart of a subject, in accordance with some applications of the invention;

FIGS. 5 and 6A-B are schematic illustrations of compressed states of implants, in accordance with some applications of the invention;

FIG. 7 is a schematic illustration of an implant in its compressed state, in accordance with some applications of the invention;

FIG. 8 is a schematic illustration of a system that comprises an implant, in accordance with some applications of the invention; and

FIGS. 9A-J are schematic illustrations of a system that comprises a plurality of subunits that are intracorporeally assembled to form an implant, in accordance with some applications of the invention.

### DETAILED DESCRIPTION OF EMBODIMENTS

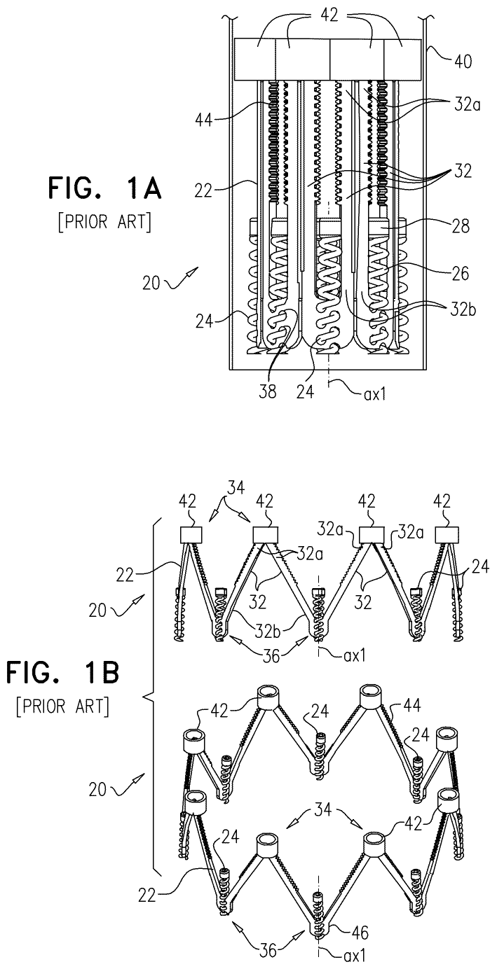

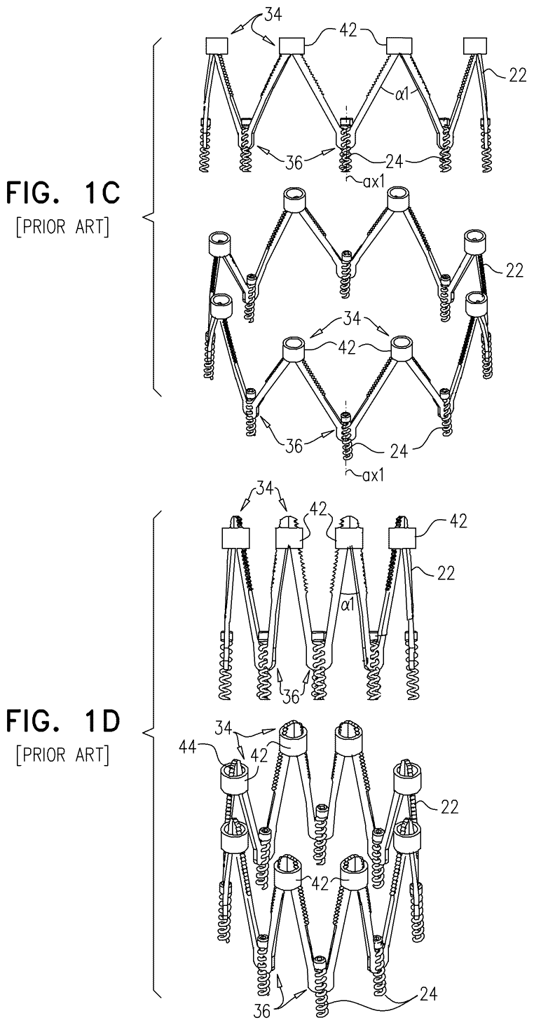

Reference is made to FIGS. 1A-D, which are schematic illustrations of a prior art implant 20 shown in U.S. Pat. No. 9,180,005, for use at a valve (e.g., a mitral valve) of a heart of a subject, in accordance with some applications of the invention. Implant 20 comprises a ring 22 and a plurality of (e.g., 8) anchors 24. Ring 22 comprises a plurality of struts 32 arranged in a pattern of alternating peaks 34 and troughs 36 (e.g., in a zig-zag pattern). Each strut 32 has a first end-portion 32a and a second end-portion 32b. Each peak 34 is defined by convergence of adjacent first end-portions 32a (i.e., of first end-portions 32a of adjacent struts 32), and each trough 36 is defined by convergence of adjacent second end-portions 32b (i.e., of second end-portions 32b of adjacent struts 32).

Each anchor 24 has a longitudinal axis ax1 along which it is configured to be driven into tissue of the annulus of the valve of the heart of the subject, and is coupled to ring 22 at a respective trough 36 in a manner that facilitates movement of the anchor along the longitudinal axis with respect to the trough. At each trough 36, ring 22 defines a plurality of holes 38 through which anchor 24 is moveable. Each anchor 24 comprises a helical tissue-engaging element 26, and an anchor head 28, and is shaped and rotatably coupled to ring 22 at the respective trough 36 such that rotation of the anchor with respect to the ring moves the anchor along its longitudinal axis with respect to the trough (e.g., corkscrews the anchor through holes 38 such that the anchor moves longitudinally). This is illustrated by FIGS. 1B-C, which show anchors 24 in a retracted position (FIG. 1B), and in an extended position after having moved along axis ax1 with respect to its respective trough 36 (FIG. 1C).

Implant 20 comprises an adjustment element 42 for each pair of adjacent first-end portions 32a. Adjustment element 42 is typically an internally-threaded nut that screws onto an external thread 44 (visible in FIG. 1D) defined by the adjacent first-end portions 32a. Such adjustment elements are actuated by rotation (e.g., using an adjustment tool, not shown), and as the adjustment elements are screwed further onto and over struts 32, the angle alpha_1 at which first end-portions 32a converge becomes smaller.

Implant 20 is an annuloplasty device, and is delivered to the heart percutaneously while in a compressed state, via a catheter 40 (FIG. 1A). Within the heart (e.g., within an atrium, such as the left atrium) implant 20 is deployed from the catheter, and automatically expands into an expanded state (FIG. 1B). While in its expanded state, implant 20 is anchored to tissue of the annulus of the valve by driving the anchors along axis ax1 and into the tissue (FIG. 1C). Typically, implant 20 is positioned such that ring 22 surrounds the orifice of the valve. Once implant 20 is anchored, it is contracted by actuating adjustment elements 42, such that angle alpha_1 is reduced (compare FIG. 1C to FIG. 1D). Contraction of implant 20 reduces (i) the circumference and the diameter of ring 22, (ii) the distance between adjacent and opposite anchors 24, and thereby (iii) the circumference and the diameter of the valve of the heart, thereby improving function of the valve.

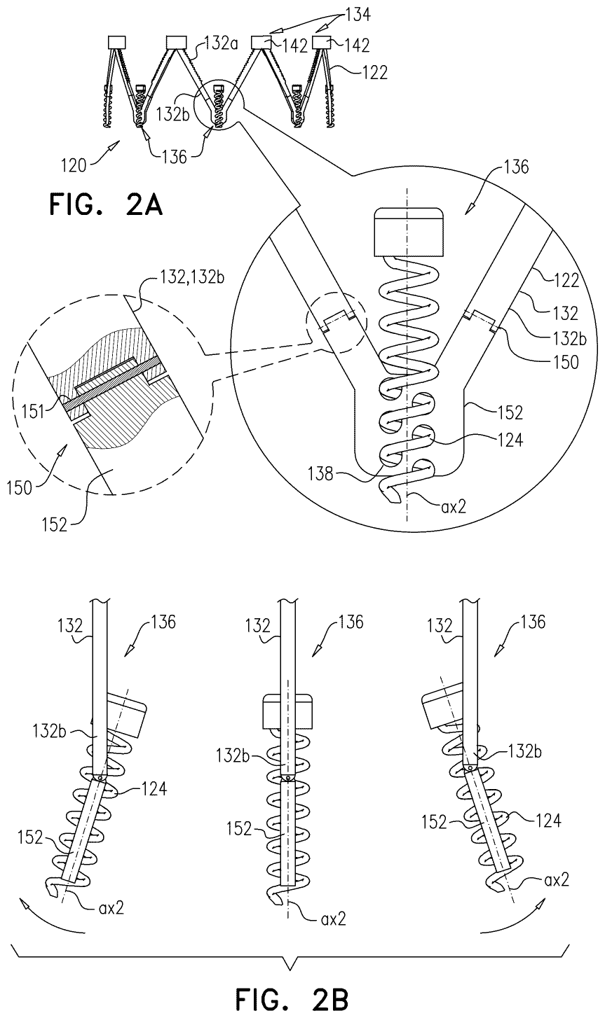

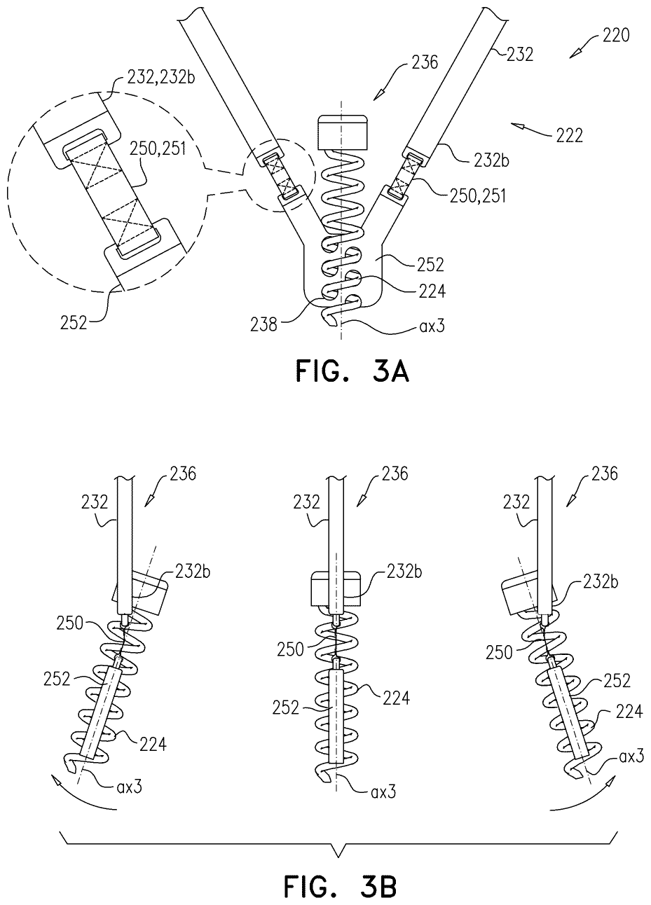

Reference is now made to FIGS. 2A-B, 3A-B, and 4, which are schematic illustrations of respective implants for use at a valve of a heart of a subject, in accordance with some applications of the invention. FIGS. 2A-B show an implant 120, FIGS. 3A-B show an implant 220, and FIG. 4 shows an implant 320. Unless noted, the structure, function, and use of implants 120, 220, and 320 are similar to those of implant 20. Unless noted, the components of implants 120, 220 and 320 generally correspond to identically-named components of implant 20, mutatis mutandis. The reference numerals assigned to the components of implants 120, 220 and 320 are intended to further illustrate this relationship. For example, struts 132 of implant 120 generally correspond to struts 32 of implant 20, mutatis mutandis (as do struts 232 of implant 220, and struts 332 of implant 320). Implants 120, 220, and 320 are typically delivered transluminally (e.g., transfemorally).

Implant 120 (FIGS. 2A-B) comprises a ring 122 and a plurality of anchors 124. Ring 122 comprises a plurality of struts 132 arranged in a pattern of alternating peaks 134 and troughs 136 (e.g., in a zig-zag pattern). Each strut 132 has a first end-portion 132a and a second end-portion 132b. Each peak 134 is defined by convergence of adjacent first end-portions 132a (i.e., of first end-portions 132a of adjacent struts 132), and each trough 136 is defined by convergence of adjacent second end-portions 132b (i.e., of second end-portions 132b of adjacent struts 132). Similarly to implant 20, implant 120 comprises an adjustment element 142 for each pair of adjacent first-end portions 132a.

Each anchor 124 has a longitudinal axis ax2 along which it is configured to be driven into tissue of the annulus of the valve of the heart of the subject, and is coupled to ring 122 at a respective trough 136 in a manner that facilitates movement of the anchor along the longitudinal axis with respect to the trough. At each trough 136, ring 122 defines a plurality of holes 138 through which anchor 124 is moveable. Typically, each anchor 124 comprises a helical tissue-engaging element and an anchor head (e.g., as described for anchor 24) and is shaped and rotatably coupled to ring 122 at the respective trough 136 such that rotation of the anchor with respect to the ring moves the anchor along its longitudinal axis with respect to the trough (e.g., corkscrews the anchor through holes 138 such that the anchor moves longitudinally).

In contrast to anchors 24 of implant 20, anchors 124 of implant 120 are coupled to ring 122 at respective troughs 136 in a manner that facilitates both (i) movement of the anchor along axis ax2 with respect to the trough, and (ii) deflection of axis ax2 with respect to the trough. That is, as well as moving axially, each anchor 124 can deflect with respect to ring 122 (e.g., with respect to struts 132 thereof). It is hypothesized by the inventors that this facilitates anchoring of implant 120 to the annulus, e.g., by allowing independent orientation of each anchor according to the tissue to which it is to be anchored.

Typically, and as shown, implant 120 (e.g., ring 122 thereof) comprises a plurality of hinges 150, at least one of which is disposed at each trough 136, and the anchor 124 disposed at that trough is coupled to ring 122 via the hinge. Hinge 150 may be a barrel hinge (e.g., comprising a pin 151, as shown), a flexure bearing, or any other suitable hinge type. For some applications, and as shown, the at least one hinge 150 of each trough 136 couples, to each other, the adjacent second end-portions 132b that define that trough. Alternatively, the adjacent second end-portions 132b may be coupled independently of the at least one hinge 150, and the at least one hinge couples anchor 124 to the trough independently of the coupling between the adjacent second end-portions (embodiment not shown).

For some applications, and as shown, implant 120 (e.g., ring 122 thereof) comprises, at each trough 136, an anchor mount 152 that is articulatably coupled to struts 132 (e.g., to second end-portions 132b), e.g., via the at least one hinge 150. Typically, each anchor mount 152 is coupled to one second end-portion 132b via one hinge 150, and to another second end-portion 132b via another hinge. Anchor mount 152 defines the holes 138 of implant 120.

FIG. 2A shows implant 120, with a magnification of a trough 136, and a further magnification of a hinge 150. FIG. 2B illustrates the articulation, at a trough 136, between ring 122 and an anchor 124 (e.g., via the articulated coupling between an anchor mount 152 and struts 132).

Implant 220 (FIGS. 3A-B) comprises a ring 222 and a plurality of anchors 224. Similar to rings 22 and 122, ring 222 comprises a plurality of struts 232 arranged in a pattern of alternating peaks (not shown) and troughs 236 (e.g., in a zig-zag pattern). Each strut 232 has a first end-portion (not shown) and a second end-portion 232b. Each peak is defined by convergence of adjacent first end-portions (i.e., of first end-portions of adjacent struts 232), and each trough 236 is defined by convergence of adjacent second end-portions 232b (i.e., of second end-portions 232b of adjacent struts 232). Similarly to implants 20 and 120, implant 220 comprises an adjustment element (not shown) for each pair of adjacent first-end portions.

Each anchor 224 has a longitudinal axis ax3 along which it is configured to be driven into tissue of the annulus of the valve of the heart of the subject, and is coupled to ring 222 at a respective trough 236 in a manner that facilitates movement of the anchor along the longitudinal axis with respect to the trough. At each trough 236, ring 222 defines a plurality of holes 238 through which anchor 224 is moveable. Typically, each anchor 224 comprises a helical tissue-engaging element and an anchor head (e.g., as described for anchor 24) and is shaped and rotatably coupled to ring 222 at the respective trough 236 such that rotation of the anchor with respect to the ring moves the anchor along its longitudinal axis with respect to the trough (e.g., corkscrews the anchor through holes 238 such that the anchor moves longitudinally).

In contrast to anchors 24 of implant 20, and similarly to anchors 124 of implant 120, anchors 224 of implant 220 are coupled to ring 222 at respective troughs 236 in a manner that facilitates both (i) movement of the anchor along axis ax3 with respect to the trough, and (ii) deflection of axis ax3 with respect to the trough. That is, as well as moving axially, each anchor 224 can deflect with respect to ring 222 (e.g., with respect to struts 232 thereof). It is hypothesized by the inventors that this facilitates anchoring of implant 220 to the annulus, e.g., by allowing independent orientation of each anchor according to the tissue to which it is to be anchored.

Typically, and as shown, implant 220 (e.g., ring 222 thereof) comprises a plurality of hinges 250, at least one of which is disposed at each trough 236, and the anchor 224 disposed at that trough is coupled to ring 222 via the hinge. Hinge 250 comprises a flexible strip 251, such as a strip of fabric. It is to be noted that although this element is named a “strip,” and is shown having a width that is greater than its thickness, and a length that is greater than its width, the term “strip” (including the specification and the claims) is not intended to limit this element to such dimensions. For some applications, and as shown, the at least one hinge 250 of each trough 236 couples, to each other, the adjacent second end-portions 232b that define that trough. Alternatively, the adjacent second end-portions 232b may be coupled independently of the at least one hinge 250, and the at least one hinge couples anchor 224 to the trough independently of the coupling between the adjacent second end-portions (embodiment not shown).

For some applications, and as shown, implant 220 (e.g., ring 222 thereof) comprises, at each trough 236, an anchor mount 252 that is articulatably coupled to struts 232 (e.g., to second end-portions 232b), e.g., via the at least one hinge 250. Typically, each anchor mount 252 is coupled to one second end-portion 232b via one hinge 250, and to another second end-portion 232b via another hinge. Anchor mount 252 defines the holes 238 of implant 220.

For some applications, hinge 250 provides a further degree of movement compared to hinge 150 of implant 120. For example, due to the flexibility of the flexible strip, anchor mount 252 may be twisted and/or deflected asymmetrically with respect to struts 232.

FIG. 3A shows a magnification of a trough 236 of implant 220 (implant 220 is not shown in its entirety), and a further magnification of a hinge 250. FIG. 3B illustrates articulation, at a trough 236, between ring 222 and an anchor 224 (e.g., via the coupling between an anchor mount 252 and struts 232).

Implant 320 (FIG. 4) comprises a ring 322 and a plurality of anchors 324. Similar to rings 22, 122, and 222, ring 322 comprises a plurality of struts 332 arranged in a pattern of alternating peaks (not shown) and troughs 336 (e.g., in a zig-zag pattern). Each strut 332 has a first end-portion (not shown) and a second end-portion 332b. Each peak is defined by convergence of adjacent first end-portions (i.e., of first end-portions of adjacent struts 332), and each trough 336 is defined by convergence of adjacent second end-portions 332b (i.e., of second end-portions 332b of adjacent struts 332). Similarly to implants 20, 120, and 220, implant 320 comprises an adjustment element (not shown) for each pair of adjacent first-end portions.

Each anchor 324 has a longitudinal axis ax4 along which it is configured to be driven into tissue of the annulus of the valve of the heart of the subject, and is coupled to ring 322 at a respective trough 336 in a manner that facilitates movement of the anchor along the longitudinal axis with respect to the trough. At each trough 336, ring 322 defines at least one hole through which anchor 324 is moveable. Typically, each anchor 324 comprises a helical tissue-engaging element and an anchor head (e.g., as described for anchor 24) and is shaped and rotatably coupled to ring 322 at the respective trough 336 such that rotation of the anchor with respect to the ring moves the anchor along its longitudinal axis with respect to the trough (e.g., corkscrews the anchor through the hole such that the anchor moves longitudinally).

In contrast to anchors 24 of implant 20, and similarly to anchors 124 of implant 120 and anchors 224 of implant 220, anchors 324 of implant 320 are coupled to ring 322 at respective troughs 336 in a manner that facilitates both (i) movement of the anchor along axis ax4 with respect to the trough, and (ii) deflection of axis ax4 with respect to the trough. That is, as well as moving axially, each anchor 324 can deflect with respect to ring 322 (e.g., with respect to struts 332 thereof). It is hypothesized by the inventors that this facilitates anchoring of implant 320 to the annulus, e.g., by allowing independent orientation of each anchor according to the tissue to which it is to be anchored.

Typically, and as shown, implant 320 (e.g., ring 322 thereof) comprises a plurality of hinges 350, each hinge disposed at a respective trough 336, and the anchor 324 disposed at that trough is coupled to ring 322 via the hinge. Hinge 350 comprises a flexible strip 351, such as a strip of fabric. For some applications, and as shown, the hinge 350 of each trough 336 couples, to each other, the adjacent second end-portions 332b that define that trough. Alternatively, the adjacent second end-portions 332b may be coupled independently of the at least one hinge 350, and the at least one hinge couples anchor 324 to the trough independently of the coupling between the adjacent second end-portions (embodiment not shown).

In contrast to implant 220, implant 320 (e.g., ring 322 thereof) typically does not comprise distinct anchor mount. Rather, anchor 324 passes directly through flexible strip 351, and the flexible strip serves as an anchor mount 352, as well as providing the articulation functionality of hinge 350. Strip 351 thereby defines the hole of each trough 336 of implant 320.

For some applications, hinge 350 provides a further degree of movement compared to hinge 150 of implant 120. For example, due to the flexibility of the flexible sheet or strip, anchor 324 may be twisted and/or deflected asymmetrically with respect to struts 332.

FIG. 4 shows a magnification of a trough 336 of implant 320 (implant 320 is not shown in its entirety). At trough 336 articulation is facilitated between ring 322 and an anchor 324.

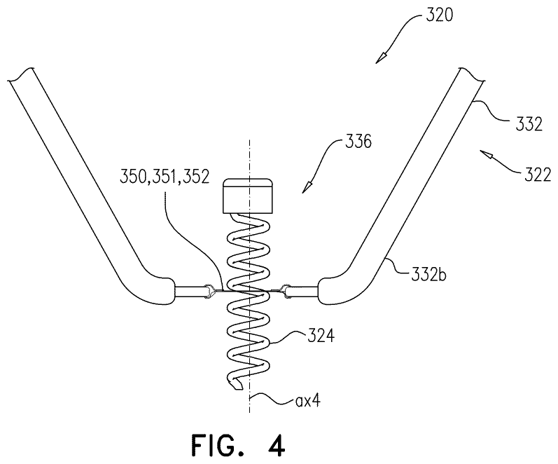

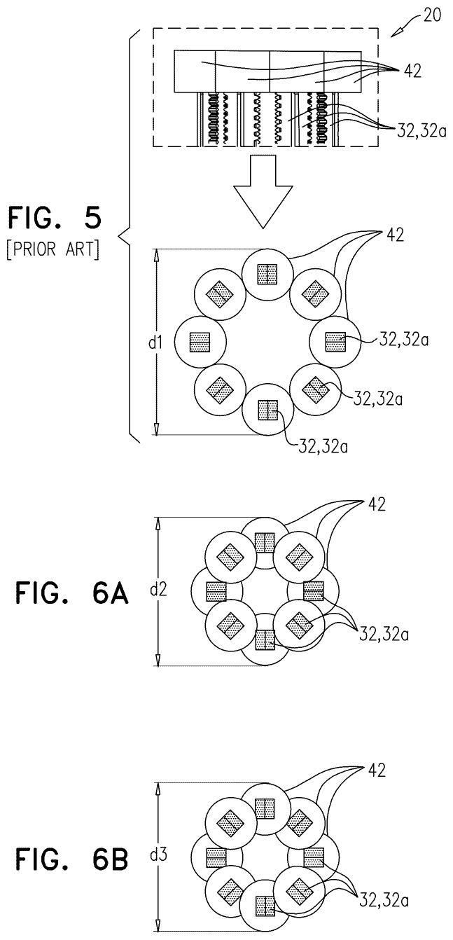

Reference is made to FIGS. 5, and 6A-B, which are schematic illustrations of compressed states of implants, in accordance with some applications of the invention. Implant 20 is used as an example, but the compressed states may apply to the other implants described herein. FIG. 5 shows a partial side view and a top view of implant 20 in its compressed state, e.g., as it would be while disposed within catheter 40, e.g., as shown in FIG. 1A. (The partial side view is taken from U.S. Pat. No. 9,180,005, whereas the top view is based on the inventors' understanding of that reference.) Adjustment elements 42 are disposed at the same longitudinal position on the implant as each other, and because they are wider than the strut-pairs to which they are coupled, they abut each other, and effectively define the widest part of implant 20 in its compressed state. The diameter of implant 20 at this widest part is shown as d1.

FIGS. 6A and 6B show alternative arrangements of adjustment elements 42 in alternative compressed states of implant 20. FIG. 6A shows adjustment elements 42 arranged in an alternating up-down pattern, and FIG. 6B shows the adjustment elements arranged in two sets of four steps. Both of these arrangements allow better compression of implant 20, such that the implant has a diameter d2 or d3 that is smaller than diameter d1. It is to be noted that diameters d2 and d3 are typically smaller than the diameter of a circle formed when all the adjustment elements 42 are arranged in a circle, touching each other.

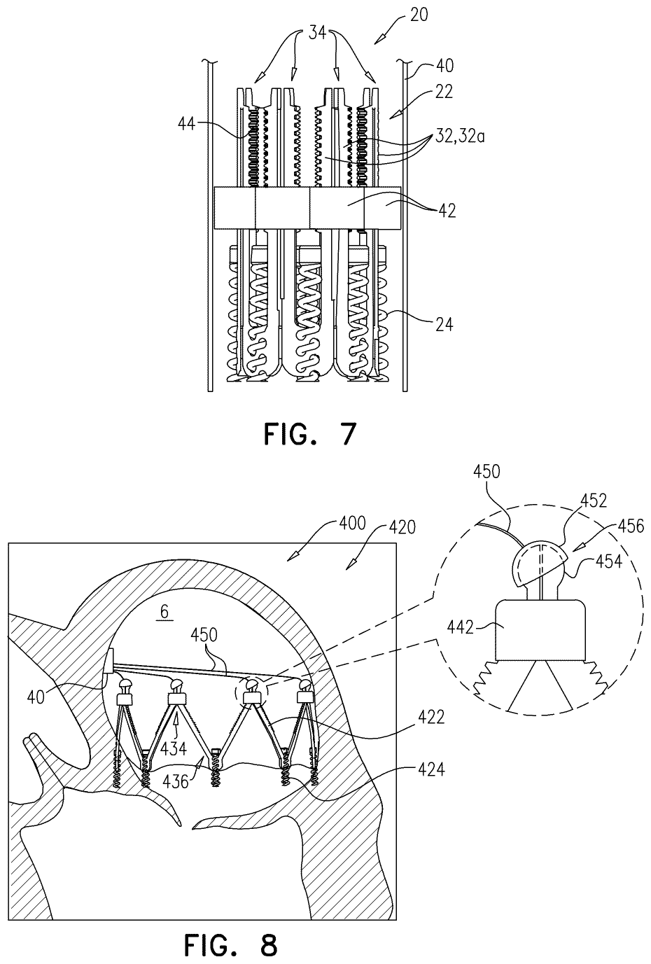

Reference is made to FIG. 7, which is a schematic illustration of an implant in its compressed state, in accordance with some applications of the invention. Implant 20 is used as an example, but the compressed state may apply to the other implants described herein. As described hereinabove, in a prior art technique, when implant 20 is deployed from catheter 40, it automatically expands into an expanded state. For such a technique, adjustment elements 42 are positioned such that they allow maximal or near-maximal expansion of ring 22 upon deployment of implant 20—e.g., at or close to peaks 34, and/or at or close to an upstream end of threads 44. In the technique of FIG. 7, adjustment elements 42 are positioned such that the restrict expansion of ring 22 upon deployment of implant 20—e.g., closer toward (e.g., at or close to) the downstream end of threads 44. Thus, upon deployment of implant 20 from catheter 40, the implant doesn't automatically expand (or at least not fully). The operator may then expand implant 20 in a controlled and/or stepwise manner by actuating adjusting elements 42 (e.g., such that they move toward peaks 34).

Reference is made to FIG. 8, which is a schematic illustration of a system 400, which comprises an implant 420, in accordance with some applications of the invention. Implant 420 comprises a ring 422 and a plurality of anchors 424. Ring 422 comprises a plurality of struts arranged in a pattern of alternating peaks 434 and troughs 436, e.g., as described herein for other implants. Similarly to other implants described herein, implant 420 comprises an adjustment element 442 for each pair of adjacent struts, e.g., disposed close to (e.g., at) each peak 434. In order to actuate (e.g., rotate) adjustment element 442, an adjustment tool is typically advanced through catheter 40 to the adjustment element. To facilitate guidance of the adjustment tool to the adjustment element, system 400 comprises a plurality of elongate guide members 450 (e.g., one for each adjustment element 442), coupled to ring 422 close to (e.g., at) each peak 434, and extending proximally into catheter 40. The adjustment tool is advanced along the elongate guide member 450 to the adjustment element 442.

Implant 420 (and the other implants described herein, including implant 20) are typically taller than annuloplasty rings known in the art, and therefore peaks 434 and adjustment elements 442 are relatively high within atrium 6. For example, peaks 434 may be only a little inferior to, at the same height as, or even superior to the site at which catheter 40 enters atrium 6 (e.g., the fossa ovalis). Each guide member 450 is coupled to ring 422 (e.g., at a respective peak 434) via a bearing 456. For example, and as shown, bearing 456 may be a ball-and-socket bearing comprising a ball 454 (e.g., defined by implant 420) and a socket 452 (e.g., coupled to, or defined by, a distal end of the guide member 450). Bearing 456 facilitates articulation between the distal end of guide member 450 and implant 420, thereby allowing adjustment elements 442 to be positioned high within atrium 6, while coupled to the guide members.

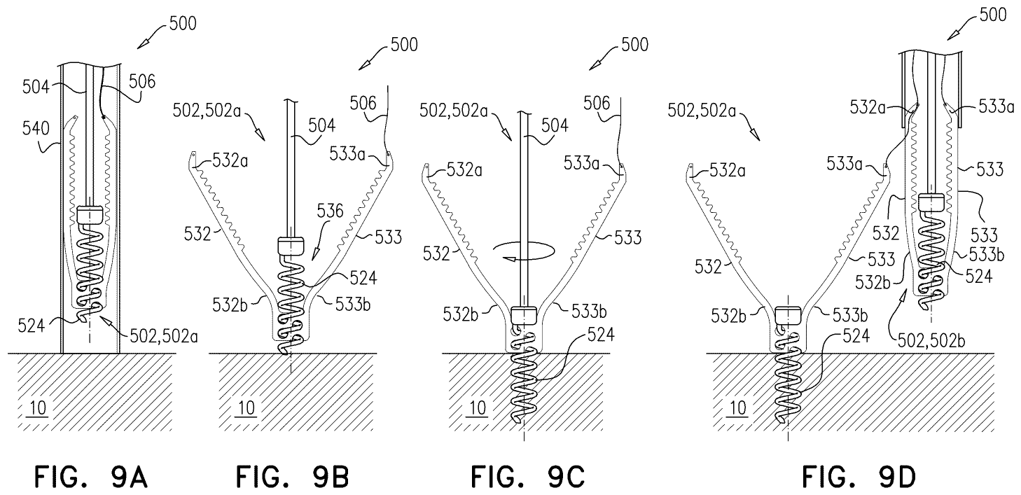

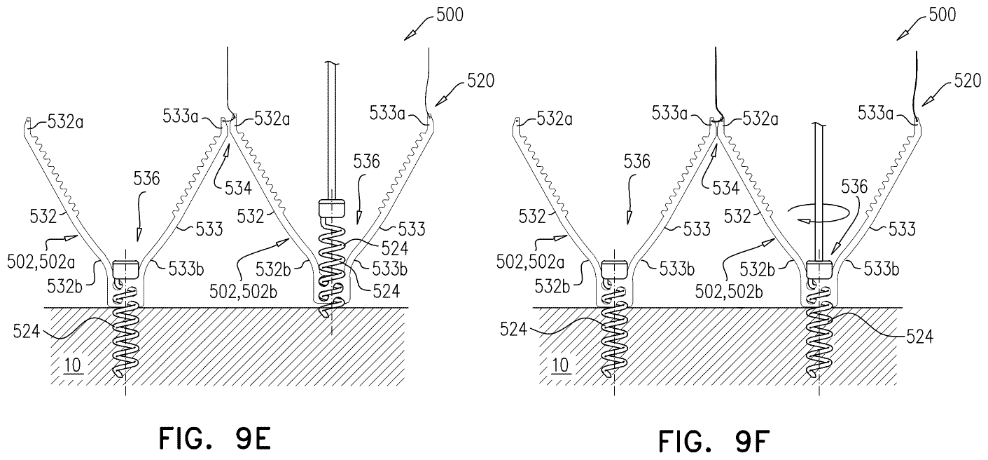

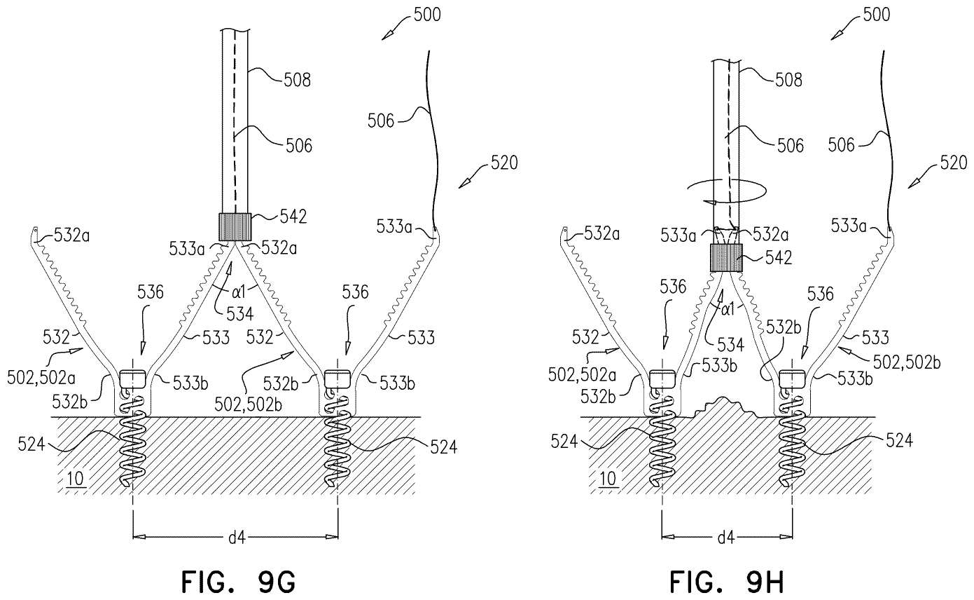

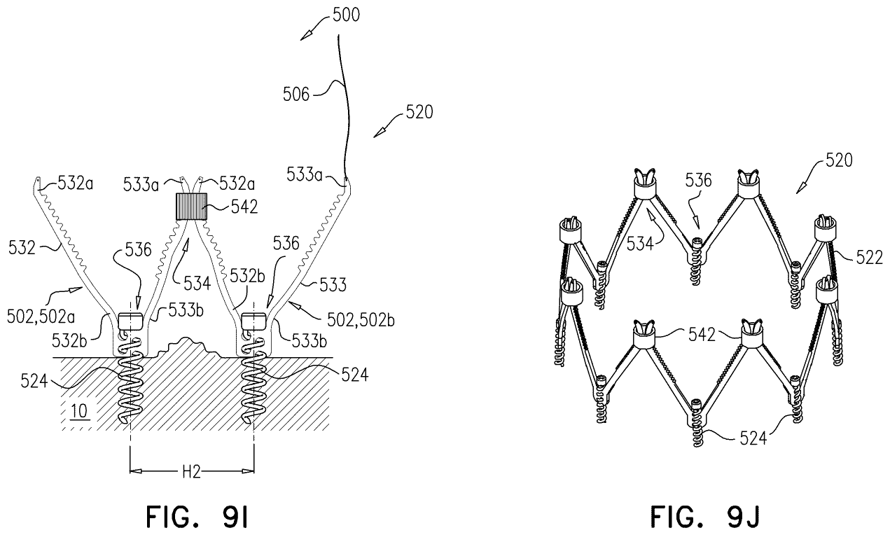

Reference is made to FIGS. 9A-J, which are schematic illustrations of a system 500, which comprises a plurality of subunits 502 that are intracorporeally assembled to form an implant 520, in accordance with some applications of the invention. Once assembled, implant 520 is similar to implant 20 and/or to another of the implants described herein, mutatis mutandis. System 500 facilitates transfemoral delivery of such an implant, by providing the implant as subunits 502, which individually have a smaller profile than that of a similar implant that is delivered pre-assembled. Thus system 500 may be considered to be a modification of any of the implants described herein, and FIGS. 9A-J may be considered to illustrate a technique for delivering such a modified implant.

Each subunit 502 comprises a pair of struts that comprises a first strut 532 (which has a first end-portion 532a and a second end-portion 532b) and a second strut 533 (which has a first end-portion 533a and a second end-portion 533b). Each subunit defines a trough 536 at which the second end-portion of each strut of the pair is coupled to the second end-portion of the other strut of the pair (i.e., end-portion 532b is coupled to end-portion 533b).

A first subunit 502a is transfemorally delivered to the native heart valve (e.g., in a compressed state, within a catheter 540), typically into an atrium such as the left atrium 6 of the heart (FIG. 9A). Subsequently, the trough 536 of subunit 502a is anchored to tissue 10 that surrounds the valve (e.g., tissue of the valve annulus) by driving a tissue anchor 524 into the tissue, such as by using an anchor driver tool 504, which may be used to facilitate deployment of the subunit out of catheter 540 (FIGS. 9B-C).

Subsequently, first end-portion 533a of second strut 533 of subunit 502a is secured to first end-portion 532a of first strut 532 of another subunit 502b, such that the secured first end-portions converge at an angle alpha_1 to define a peak 534 (FIGS. 9D-G). For example, and as shown, end-portion 533a of first subunit 502a may have an elongate guide member 506 attached thereto, the guide member extending proximally from end-portion 533a of the implanted subunit (e.g., into a delivery sheath, such as to outside of the subject), and end-portion 532a of subunit 502b is advanced over and along the guide member, guided by the guide member to end-portion 533a of subunit 502a. Subunit 502b is anchored to tissue 10 in the same way as subunit 502a.

Typically, an adjustment element 542 is subsequently coupled to the first end-portions that define each peak (FIG. 9G). For some applications, this further secures these two end-portions to each other. For example, for applications in which adjustment element 542 comprises a nut (e.g., as described hereinabove, mutatis mutandis), the nut is typically screwed onto the threads defined by the end-portions (e.g., using a tool 508 that is reversibly coupled to the adjustment element, and is slidable over and along guide member 506), thereby securing the end-portions to each other.

Subsequently to the steps of anchoring and securing, the angle alpha_1 defined by each peak 534 is reduced by actuating the adjustment element 542 of that peak, e.g., using tool 508. This reduces a distance d4 between anchors 524 of the adjacent subunits, thereby reducing the circumference of a portion of the annulus of the valve being treated. The adjustment is shown in FIG. 9H in order to illustrate that the adjustment of each adjustment element 542 may be performed after each subunit 502 is secured. Alternatively, the adjustment may be deferred until after more than one (e.g., all) of the subunits have been secured (e.g., similarly to implant 20).

Following implantation, guide member 506 is typically decoupled from implant 520 and removed from the subject (FIG. 9I). This may be achieved by unthreading and/or cutting each guide member 506 (e.g., facilitated by tool 508), or by any other suitable technique known in the art.

The above process is repeated iteratively, mutatis mutandis, until implant 520 has been fully assembled, e.g., formed into a full ring such as that of implant 20, or into a partial ring or band (FIG. 9J).

The techniques described with reference to FIGS. 9A-J may be used to assemble implants similar to implants 20, 120, 220 and 320, mutatis mutandis.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for use with a valve of a heart of a subject, the method comprising:
assembling an annuloplasty implant at the valve by:
transfemorally delivering, to the heart, a first subunit of the annuloplasty implant;
securing the first subunit to tissue of the heart by driving, into the tissue, a first anchor connected to the first subunit by a flexible strip; and
subsequently, for multiple successive subunits of the annuloplasty implant, iteratively:
transfemorally delivering, to the heart, the successive subunit; and
securing the successive subunit to the tissue of the heart by driving, into the tissue, a respective anchor connected to the successive subunit by a respective flexible strip; and
subsequently, contracting the valve by contracting the annuloplasty implant.

2. The method according to claim 1, wherein assembling the annuloplasty implant comprises forming a ring from the first and successive subunits.

3. The method according to claim 1, wherein:
for each of the successive subunits, the respective flexible strip is a fabric strip, and
securing the successive subunit to the tissue of the heart comprises securing the successive subunit to the tissue of the heart by driving, into the tissue, the respective anchor connected to the successive subunit by the respective fabric strip.

4. The method according to claim 1, wherein, for each of the successive subunits, transfemorally delivering the successive subunit to the heart comprises transfemorally advancing the successive subunit using a driver coupled to the respective anchor.

5. The method according to claim 1, further comprising advancing an adjustment tool to the heart, wherein contracting the annuloplasty implant comprises contracting the annuloplasty implant using the adjustment tool.

6. The method according to claim 5, wherein:
advancing the adjustment tool to the heart comprises advancing the adjustment tool to engage one of the successive subunits subsequently to assembling the annuloplasty implant, and
contracting the annuloplasty implant comprises contracting the annuloplasty implant while the adjustment tool remains engaged with the one of the successive subunits.

7. The method according to claim 1, wherein, for each of the successive subunits, transfemorally delivering the successive subunit to the heart comprises transfemorally advancing the successive subunit over and along a guide member to which the preceding subunit of the annuloplasty implant is coupled.

8. The method according to claim 1, further comprising, for at least one of the successive subunits, deflecting the respective anchor with respect to the annuloplasty implant.

9. The method according to claim 1, wherein, for each of the successive subunits, driving the respective anchor into the tissue comprises screwing the respective anchor into the tissue by rotating the respective anchor with respect to the flexible strip.

10. The method according to claim 9, wherein, for each of the successive subunits, driving the respective anchor into the tissue comprises screwing the respective anchor through the respective strip and into the tissue.
